(19) United States
(12) Patent Application Publication
(10) Pub. No.: US 2023/0277113 A1
(43) Pub. Date: Sep. 7, 2023

(54) HEADSET WITH WIRELESS ELECTROENCEPHALOGRAPH FOR NEURAL CONDITIONING

(71) Applicant: Kamran Fallahpuor, New York, NY (US)

(72) Inventor: Kamran Fallahpuor, New York, NY (US)

(21) Appl. No.: 17/687,016

(22) Filed: Mar. 4, 2022

(51) Int. Cl. A61B 5/369; A61B 5/00; A61B 5/291; A61B 5/251

(52) U.S. Cl. CPC A61B 5/369; A61B 5/6803; A61B 5/291; A61B 5/0006; A61B 5/7267; A61B 5/251; A61B 2560/04

(57) ABSTRACT

A headset includes a wireless encephalograph that extends from front to back over said human's head and that has electrodes. The electrodes are disposed such that when a first electrode is disposed over a first site on the human's head, the second and third electrodes are disposed over corresponding second and third sites on said human's head, wherein the first, second, and third sites are selected from the group consisting of said human's posterior cortex, anterior cortex, and motor/sensory cortex.

### BACKGROUND

A human brain comprises neurons that engage in electrical activity. This electrical activity can be detected non-invasively by placing electrodes on the scalp. The resulting waveforms, referred to colloquially as “brain waves,” provide some insight into the human's mind. Changes in these brain waves thus provide a way to detect a state change in the human's mind.

For example, certain types of brain waves are known to be associated with sleep or extreme relaxation. Disturbances in these brain waves thus imply that a subject may have become aroused. Other types of brain waves are known, from observation, to be indicative of a state of attention. Changes in such brain waves thus provide a basis for inferring that a subject may have lapsed into a state of distraction.

A human subject has some control over such state changes and may attempt to essentially will oneself to focus or to lull oneself into a state of relaxation.

However, a human's ability to control state-of-mind is limited and also varies widely across the population. There are many people whose ability to transition into and remain in a state of attention is quite limited. In some cases, this inability is sufficiently extreme such that pharmaceuticals are used to artificially induce the desired state. There are also large swaths of the population that have difficulty relaxing. Again, such people often resort to pharmaceuticals in order to reach the desired state of consciousness.

### SUMMARY

In one aspect, the invention features a brain-training system for training neurons in a human's head to transition into a desired state. The brain-training system includes a headset that includes an electroencephalograph that extends an arcuate path in a median plane of the human's head. The electroencephalograph includes and first, second, and third electrodes, all of which are dry electrodes. The second and third electrodes are disposed relative to the first electrode such that, when the first electrode is disposed over a first site on the human's head, the second and third electrodes are disposed over corresponding second and third sites on the human's head. The first, second, and third sites are selected from the group consisting of the human's posterior cortex, anterior cortex, and motor/sensory cortex.

In some embodiments, the headset includes earcups within which are loudspeakers for providing a conditioning stimulus for the neurons. When the earcups are over the human's ears the first electrode is disposed over the first site. Among these are embodiments in which at least one earcup includes a grounding electrode.

In other embodiments, the headset includes a headband extends along an arcuate path in the head's coronal plane and loudspeakers disposed at ends of the headband. Among these are embodiments in which the headband is integral with the electroencephalograph and those in which it is configured to transition between being attached to the headband and being detached from the headband.

Also among the embodiments are those in which the electrodes comprise protruding pins that are configured to make contact with the human's scalp. Among these are embodiments in which the pins resist being pushed back by a restoring force. This restoring force is one that increases with the to which the pins are pushed back. Examples of suitable pins are spring-loaded pins.

In still other embodiments, each of the electrodes includes a silicone layer and conductive pins that extend through the silicone layer.

To accommodate heads of different sizes and shapes, some embodiments feature a flexible electroencephalograph to permit the electrodes to move relative to each other.

Embodiments further include those in which the electroencephalograph includes a wireless interface for transmitting a measured signal. This measured signal is one that has been derived from a signal obtained from the electrodes. Still other embodiments include a multiplexer that selects a signal from the electrodes for conversion into a digital signal.

Other embodiments of the brain-training system further include a training application executing on a portable device. The training application is one that is configured for wireless communication with the headset so as to both receive measurement signals from the electroencephalograph and transmit a conditioning stimulus to the headset.

Also among the embodiments are those in which the brain-training system further includes remote circuitry. Among these are embodiments in which the remote circuitry provides a series of conditioning stimuli in response to measurement signals received from real-time monitoring by the electroencephalograph, those in which the remote circuitry includes feature-extraction circuitry that extracts features from measurement signals that result from real-time monitoring by the electroencephalograph, and those in which the electroencephalograph is one of a plurality of encephalographs that are all in communication with the remote circuitry. In this latter embodiment, for each of the electroencephalographs, the remote circuitry receives a measurement signal and transmits, in response, a conditioning stimulus. This conditioning stimulus is one that has been tailored to cause features extracted from the measurement signal to move towards features in a target feature-set that corresponds to a desired mental state that is provided by a user of the electroencephalograph.

Also among the embodiments that include remote circuitry are those in which the remote circuitry provides a conditional stimulus in response to measurement signals received from real-time monitoring by the electroencephalograph. In such cases, the conditional stimulus is selected to cause the neurons to generate a measurement signal having a measured feature-set that approximates a target feature-set.

Also among the embodiments are those in which the headset further includes loudspeakers, and the brain-training system further includes a portable device and remote circuitry that provides a conditional stimulus in response to measurement signals received from the portable device. These measurement signals include information obtained from real-time monitoring by the electroencephalograph. The conditional stimulus includes both an audio constituent that is to be played on the loudspeakers and a video constituent that is to be displayed on the portable device. The portable device executes a training application that separates the audio constituent from the video constituent and forwards the audio constituent to the headset to be played on the loudspeakers.

In still other embodiments, the brain-training system further includes remote circuitry that provides a conditional stimulus in response to received measurement signals. In such embodiments, the conditional stimulus includes an audio constituent that is to be exposed to the neurons. This audio constituent includes a music component and a reward component.

Additional embodiments of a brain-training system having remote circuitry include those in which remote circuitry provides a conditional stimulus in response to received measurement signals. In such embodiments, the conditional stimulus includes an audio constituent that is to be exposed to the neurons. This audio constituent includes music that includes a weighted sum of music components that is modified by the remote circuitry based on changes in the received measurement signals.

In some embodiments, the remote circuitry includes application-specific circuitry that includes resistors, capacitors, inductors, transistors, and diodes together with a clock that controls the intervals in which charge is made to move through the various circuit elements. Among the circuit elements are arrays of semiconductor devices that maintain one of two desired states over time and that are made to transition between states at selected times.

The various steps carried out by the remote circuitry have proven to be incapable of being performed in a human mind given its current state of evolution. Indeed, it was for this reason that remote circuitry was required to implement the methods described herein.

Additionally, the various steps carried out by the remote circuitry have proven to be incapable of being performed have also been found to be incapable of being on a generic computer. Thus far, they have only been performed on a non-generic computer.

All attempts to cause the remote circuitry to perform the methods described herein in an abstract manner have thus far failed. Each attempt resulted in performance of the method in a non-abstract manner, where “non-abstract” is defined herein as the converse of “abstract” as that term is used by the Supreme Court of the United States.

The claims are explicitly defined to include only non-abstract implementations of the recited apparatus and methods, where “non-abstract” has been defined as above. Any party who presumes to construe the claims as being abstract in nature would simply be proving that it is possible to improperly construe the claims in a manner inconsistent with express statements to the contrary within the specification.

These and other features will be apparent from the following detailed description and the accompanying figures, in which:

### DESCRIPTION OF DRAWINGS

FIG. 1 shows a brain-training system comprising a headset;

FIG. 2 shows details of the headset shown in FIG. 1;

FIG. 3 shows circuitry in the headset shown in FIG. 1; and

FIG. 4 shows constituents of a stimulus generated by the brain-training system of FIG. 1.

### DETAILED DESCRIPTION

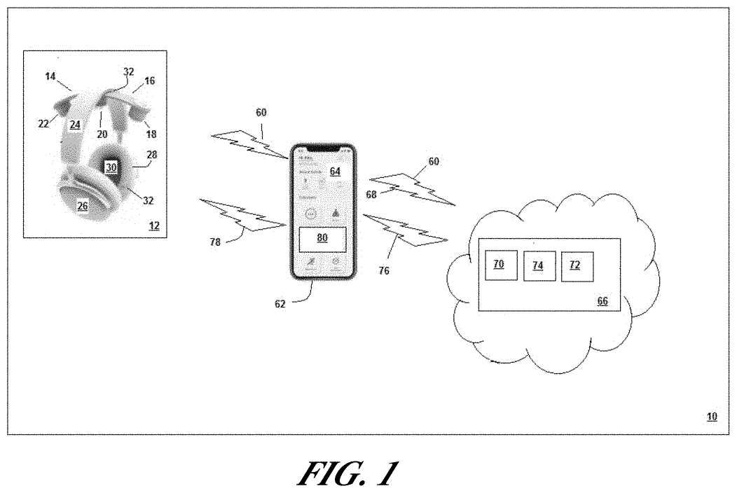

FIG. 1 shows a brain-training system 10 for conditioning neurons in the brain of a subject. The brain-training system 10 conditions the neurons into cooperating to cause a transition into a desired mental state and to thereafter cause the neurons to remain in that mental state. Examples of mental states include a relaxed state, a stress-reducing state, a state conducive to sleep, a state conducive to enhancement of focus, and a state conducive to greater attention.

For ease of discussion, it will be useful to define certain directions relative to a subject's head. Accordingly, as used herein, a “median plane” is a plane that bisects the head along the corpus callosum into left and right hemispheres. As used herein, a “transverse plane” is a plane that is perpendicular to the medial plane and passes through the ears. The “coronal plane” is one that is perpendicular to the transverse and median planes. A “path” in any of the foregoing planes is a continuous set of points in that plane having two endpoints.

The training system 10 includes a headset 12 that comprises an electroencephalograph 14. The electroencephalograph 14 includes a housing 16 that extends along an arcuate path in the median plane so as to roughly follow the contour of the head along a medial direction. The housing 16 supports electrodes 18, 20, 22. These are dry electrodes that do not require a conductive gel to make contact. In some embodiments, the housing is rigid. However, in others, the housing is flexible and thus adjustable to more closely follow the contour of the head along the medial direction.

In a preferred embodiment, the headset 12 also comprises a headband 24 that stabilizes the electroencephalograph 14. The headband 24 extends along an arcuate path in the coronal plane so as to follow the contour of the head along the direction in which the coronal plane extends. In addition to its role as a stabilizer for the electroencephalograph 14, the headband 24 also supports first and second earcups 26, 28 at ends thereof. Each earcup 26, 28 comprises a loudspeaker 30 that is to be used in connection with neural conditioning. At least one earcup comprises a ground contact 32 to provide a reference voltage for the electrodes 18, 20, 22.

In some embodiments, a fastener 34 provides a mechanical coupling between the housing 16 and the headband 24. As a result, the headband 24 can be separated from the headset 12 and later reattached to the headset 12. In other embodiments, the housing 16 is integral with the headset 12 and therefore cannot be detached from the headset 12. In either case, the headband 24 and the housing 16 are positioned relative to each other such that when a subject wears the headset 12, each electrode 18, 20, 22 makes contact with a corresponding site of the subject's scalp so as to provide real-time monitoring of brain waves emanating from that site.

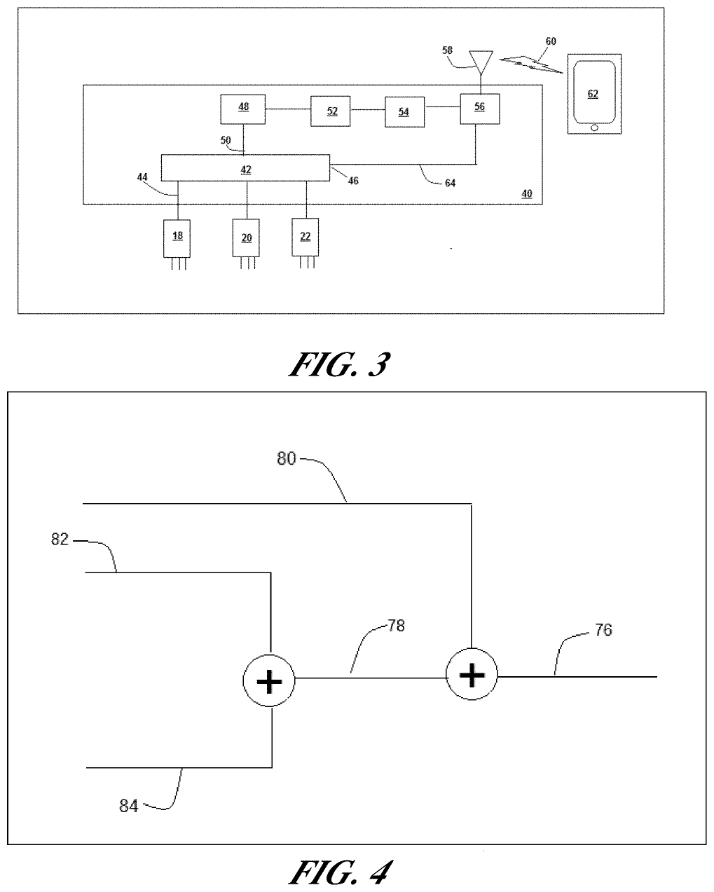

The sites are selected based on the mental state that the subject's neurons are to be trained to achieve. In particular, the sites are chosen to be adjacent to the locations of neurons that are known to be pertinent to assessing the existence of a selected mental state. The three sites shown in FIG. 3 are the posterior cortex, the anterior cortex, and the motor/sensory cortex. Such placement is useful for conditioning the brain to transition into states of greater attention, focus, and relaxation, respectively. Suitable placements, using the International 10-20 system, are Fz, Cz, and Pz.

The earcups 26, 28 serve as fiducials for correct placement of the electrodes 18, 20, 22. In particular, the housing 16 is disposed on the headset 12 such that when the earcups 26, 28 are in the correct position over the subject's ears, the electrodes 18, 20, 22 will be disposed over the correct sites on the subject's scalp. In the illustrated embodiment, the electrodes 18, 20, 22 are placed relative to each other so that if one is disposed over the first site, the other two are disposed over the second and third sites respectively. Each electrode 18, 20, 22 receives brain waves from the corresponding sites over which it is disposed. In general, it is not necessary to activate all three electrodes.

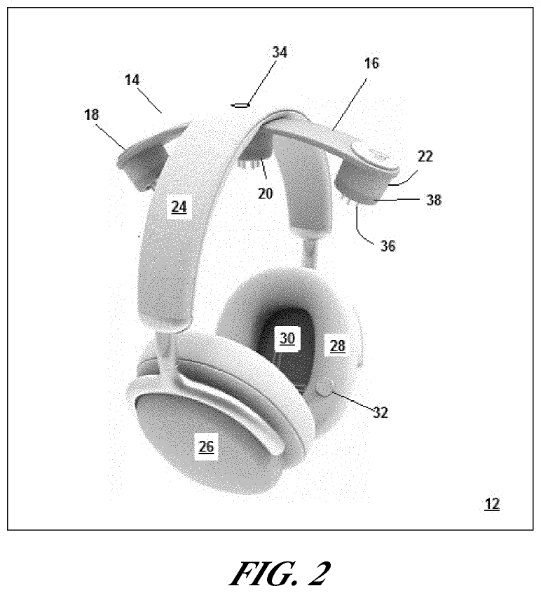

Because hair is often found on the scalp, it is useful for each electrode 18, 20, 22 to comprise pins 36 that extend towards the subject's scalp, as shown in FIG. 2. In a preferred embodiment, the pins 36 are biased to project towards the scalp. In such cases, each pin 36 resists a force that tends to reduce the extent to which it projects towards the scalp. Among these embodiments are those in which the pins 36 are spring-loaded pins 36. To provide a more comfortable fit, it is also useful for the pins 36 to protrude from a flexible layer 38. In a typical embodiment, the flexible layer 38 comprises silicone.

Referring now to FIG. 3, the electroencephalograph 14 features circuitry 40 that comprises a multiplexer 42 for receiving analog signals 44 from the electrodes 18, 20, 22. The multiplexer 42 also includes a selection input 46 for selecting which of the analog signals 44 is to be passed to an analog-to-digital converter 48. Embodiments include those in which the selected signal is that of only one electrode 18, 20, 22. However, in some embodiments, the multiplexer 42 combines weighted outputs from two or more electrodes 18, 20, 22, thereby effectively creating a synthetic electrode.

The analog-to-digital converter 48 samples the analog signal 44 and quantizes it into discrete levels to form a corresponding digital signal 50. The resulting digital signal 50 is then provided to noise-reduction circuitry 52 and filtering circuitry 54 before being provided to a wireless interface 56 for transmission via an antenna 58 as a measurement signal 60 that is ultimately received by a portable device 62. An example of a portable device 62 is a smartphone, a tablet, smart jewelry, such as a smart watch, or a personal computer.

A training application 64 executing on the portable device 62 will have received from the subject instructions indicative of what mental state the training system 10 should attempt to achieve.

The wireless interface 56 receives a selection signal 64 from the portable device 62. The selection signal 64 selects the appropriate electrode for use in real-time monitoring. The circuitry 40 causes this selection signal 64 to be provided to the selection input 46 of the multiplexer 42.

Referring back to FIG. 1, the training application 64 uses the digital signals 18 received from the electroencephalograph 14 to display brain waves in real time on the portable device 62. In addition, the training application 64 causes the portable device 62 to forward the measurement signal 60 to remote circuitry 66 in the cloud together with desired-state information 68 representative of what sort of neural conditioning the subject wishes to achieve.

The remote circuitry 66 includes feature-extraction circuitry 70 carries out feature extraction on the measurement signal 60 to obtain measured feature-set 72 for the subject. Based on the desired-state information 68, the remote circuitry 66 defines a target feature-set 74.

The remote circuitry 66 then formulates a conditioning stimulus 76 to which the subject's neurons are to be exposed to begin the conditioning process. The conditioning stimulus 76 is tailored to cause the features present in the baseline to transition into the target features.

Referring to FIG. 4, the conditioning stimulus 76 comprises an audio constituent 78 and a video constituent 80 to which the subject is to be exposed in an attempt to condition the relevant neurons so that they achieve and maintain the desired brain state.

The remote circuitry 66 transmits the conditioning stimulus 76 to the training application 64, together with synchronizing information to ensure that the audio constituent 78 and video constituent 80 are displayed at the correct times relative to each other. Upon receipt of the conditioning stimulus 76, the training application 64 separates the video constituent 80 from the audio constituent 78 and displays the video constituent 80 on the portable device 62. The training application 64 then sends the audio constituent 78 to the headset 12 to be listened to by the subject. As a result, the subject's neurons are exposed to the conditioning stimulus 76 using different sensory pathways.

The training application 64 continues to receive a measurement signals 60 from the electroencephalograph 14 as the subject is exposed to the conditioning stimulus 76. These updated measurement signals 60 are then transmitted to the remote circuitry 66 to serve as a basis for feedback control over the neural conditioning process.

The remote circuitry 66 carries out further feature extraction on the updated measurement signal 60. The resulting updated measured feature-sets 72 provide a basis for evaluating the effect of the conditioning stimulus 76 and, in particular, the progress made towards driving the measured feature-set 72 towards the target feature-set 74. In response to the assessment of such progress, the remote circuitry 66 then formulates a revised conditioning-stimulus 76. It then transmits the revised conditioning-stimulus 76 back to the training application 64 so that the neurons to be conditioned can be exposed to them via the subject's sensory pathways.

The training system 10 thus forms a distributed closed-loop feedback system that attempts to guide the subject's brain waves to achieve a particular feature set through exposure to conditioning stimulus 76, with the conditioning stimulus 76 being adapted periodically in an effort to guide the received feature set towards the target feature set.

In some embodiments, the audio constituent 78 comprises a superposition of a music component 82 and a reward component 84. The reward component 84 is made to appear or disappear or is otherwise altered in response to the progress being made towards arriving at the target feature set. In some embodiments, the reward component 84 is a single tone whereas in others it is a combination of frequencies.

The music component 82 itself can be viewed as a superposition of components. The remote circuitry 66 would therefore be able to also vary the audio constituent 78 of the conditioning stimulus 76 by modifying this superposition of the music's components.

In some cases, the music's components form an orthogonal basis of a function space. For example, the components can be complex exponentials such as those used in a Fourier transform. In such cases, the remote circuitry 66 adaptively varies the conditioning stimulus 76 to suppress or enhance certain frequencies of the music component 82 in an attempt to drive the brain waves to have the desired feature set.

In other cases, the components of the music component 82 do not form an orthogonal basis of the function space. For example, a first component could be the function that, when played by itself, contains the sounds made by the string section and a second component could be the function that, when played by itself, sounds the rest of the orchestra minus the string section from the first component. This granularity of components can be further increased. For example, the components may include a function that contains the sound played by a particular violin.

In either case, the components whose superposition forms the music component 82 can be individually weighted by a complex number so as to modify the amplitude of that component and its phase relative to other components in an attempt to tune the conditioning stimulus 76 to drive the features obtained from the measurement signal 60 towards the target feature. In effect, this generalizes the concept of the reward tone 84 from being restricted to a drone-like sound to a more general transformation of the components of a musical composition.

In still other embodiments, either the audio or video constituent 78, 80 of the conditioning stimulus 76 is adaptively modified based on changes in brain state or in neural activity. Examples include causing the music component 82 to pause, by changing the overall volume of the music component 82 as a whole or on a component-by-component basis, or by changing the perceived source of the audio constituent, for example by varying the relative volumes of the loudspeakers 30.

It should be noted that the act of modifying an existing musical composition by assigning weights to its components can be viewed as effectively creating a new composition. As a result, the remote circuitry 66 can be viewed as adaptively composing a music component 82 in an effort to condition neurons in the subject's brain to achieve a desired state, the desired state having been defined by the target features.

Having described the invention and a preferred embodiment thereof, what is claimed as new and secured by letters patent is:

What is claimed is:

1. An apparatus comprising a brain-training system for training neurons in a human's head to transition into a desired state, said brain-training system comprising a headset that comprises an electroencephalograph that extends an arcuate path in a median plane of said human's head, wherein said electroencephalograph comprises and first, second, and third electrodes, wherein said electrodes are dry electrodes, wherein said second and third electrodes are disposed relative to said first electrode such that, when said first electrode is disposed over a first site on said human's head, said second and third electrodes are disposed over corresponding second and third sites on said human's head, and wherein said first, second, and third sites are selected from the group consisting of said human's posterior cortex, anterior cortex, and motor/sensory cortex.

2. The apparatus of claim 1, wherein said headset comprises earcups comprising loudspeakers for providing a conditioning stimulus for said neurons, wherein when said earcups are over said human's ears, said first electrode is disposed over said first site.

3. The apparatus of claim 1, wherein said headset comprises a headband extends along an arcuate path in a coronal plane of said head and loudspeakers disposed at ends of said headband.

4. The apparatus of claim 1, wherein said headset comprises a headband that is integral with said electroencephalograph.

5. The apparatus of claim 1, wherein said headset comprises a headband that is configured to transition between being attached to said headband and being detached from said headband, said headband extending along said head's coronal plane.

6. The apparatus of claim 1, wherein said electrodes comprise protruding pins configured to make contact with said human's scalp.

7. The apparatus of claim 1, wherein said electrodes comprise pins that resist being pushed back by a restoring force that increases as an extent to which said pins are pushed back increases.

8. The apparatus of claim 1, wherein each of said electrodes comprises a silicone layer and conductive pins that extent through said silicone layer.

9. The apparatus of claim 1, wherein said electroencephalograph is flexible so as to permit said electrodes to move relative to each other.

10. The apparatus of claim 1, wherein said headset comprises an earcup and an electrode on said earcup.

11. The apparatus of claim 1, wherein said electroencephalograph comprises a wireless interface for transmitting a measured signal derived from a signal obtained from said electrodes.

12. The apparatus of claim 1, wherein said electroencephalograph comprises a multiplexer that selects a signal from said electrodes for conversion into a digital signal.

13. The apparatus of claim 1, wherein said brain-training system further comprises a training application executing on a portable device, said training application being configured for wireless communication with said headset to receive measurement signals from said electroencephalograph and to transmit a conditioning stimulus to said headset.

14. The apparatus of claim 1, wherein said brain-training system further comprises remote circuitry that provides a series of conditioning stimuli in response to measurement signals received from real-time monitoring by said electroencephalograph.

15. The apparatus of claim 1, wherein said brain-training system further comprises feature-extraction circuitry that extracts features from measurement signals that result from real-time monitoring by said electroencephalograph.

16. The apparatus of claim 1, wherein said brain-training system further comprises remote circuitry, wherein said electroencephalograph is one of a plurality of encephalographs that are all in communication with said remote circuitry, wherein, for each of said electroencephalographs, said remote circuitry receives a measurement signal and transmits, in response, a conditioning stimulus, said conditioning stimulus having been tailored to cause features extracted from said measurement signal to move towards features in a target feature-set that corresponds to a desired mental state that is provided by a user of said electroencephalograph.

17. The apparatus of claim 1, wherein said brain-training system further comprises remote circuitry that provides a conditional stimulus in response to measurement signals received from real-time monitoring by said electroencephalograph, said conditional stimulus being selected to cause said neurons to generate a measurement signal having a measured feature-set that approximates a target feature-set.

18. The apparatus of claim 1, wherein said headset further comprises loudspeakers, wherein said brain-training system further comprises a portable device and remote circuitry, wherein said remote circuitry provides a conditional stimulus in response to measurement signals received from said portable device, said measurement signals comprising information obtained from real-time monitoring by said electroencephalograph, wherein said conditional stimulus comprises an audio constituent that is to be played on said loudspeakers and a video constituent that is to be displayed on said portable device, and wherein said portable device executes a training application that separates said audio constituent from said video constituent and forwards said audio constituent to said headset to be played on said loudspeakers.

19. The apparatus of claim 1, wherein said brain-training system further comprises remote circuitry, wherein said remote circuitry provides a conditional stimulus in response to received measurement signals wherein said conditional stimulus comprises an audio constituent that is to be exposed to said neurons, wherein said audio constituent comprises a music component and a reward component.

20. The apparatus of claim 1, wherein said brain-training system further comprises remote circuitry, wherein said remote circuitry provides a conditional stimulus in response to received measurement signals wherein said conditional stimulus comprises an audio constituent that is to be exposed to said neurons, wherein said audio constituent comprises music that comprises a weighted sum of music components, and wherein said remote circuitry is configured to modify said weighted sum based on changes in said received measurement signals.

21. The apparatus of claim 1, wherein said brain-training system further comprises remote circuitry, wherein said remote circuitry provides a conditional stimulus in response to received measurement signals wherein said conditional stimulus comprises a video constituent that is to be exposed to said neurons, remote circuitry is configured to modify said video constituent based on changes in said received measurement signals.
